(19) United States
(12) Patent Application Publication
(10) Pub. No.: US 2010/0191335 A1
(43) Pub. Date: Jul. 29, 2010

(54) SPINAL NUCLEUS PROSTHESIS DEVICE

(75) Inventors: Jonathan Root, San Francisco, CA (US); Alon Shalev, Raanana (IL); Alexander Dubson, Petach-Tikva (IL); Oded Nissan, Modiin (IL)

Correspondence Address: BROWDY AND NEIMARK, P.L.L.C.;624 NINTH STREET, NW, SUITE 300, WASHINGTON, DC, 20001-5303, US

(73) Assignee: NICAST LTD., Lod (IL)

(21) Appl. No.: 12/436,050

(22) Filed: May 5, 2009

Related U.S. Application Data
(63) Continuation of application No. 12/094,540.
(60) Provisional application No. 60/738,045, filed on Nov. 21, 2005.

(51) Int. Cl. A61F 2/44

(52) U.S. Cl. 623/17.16

(57) ABSTRACT

An implantable prosthesis device is disclosed. The prosthesis device comprises one or more compartments bounded by a substantially closed porous envelope made at least in part of non-woven polymer fibers, and a filler structure at least partially filling the compartment(s) and being made, at least in part, of non-woven swellable polymer fibers.

### FIELD AND BACKGROUND OF THE INVENTION

The present invention relates to bioartificial implants and, more particularly, but not exclusively, to an implantable prosthesis suitable for replacing or augmenting the nucleus pulposus tissue.

The human spinal column comprises a plurality of articulating bony elements (vertebrae) separated by soft tissue intervertebral discs. The intervertebral discs are flexible joints which provide for flexion, extension and rotation of the vertebrae relative to one another, thus contributing to the stability and mobility of the spine within the axial skeleton. At the center of each intervertebral disc there is a gelatinous material, known as the nucleus pulposus, which in adults is composed of cells and an insoluble extracellular matrix produced by the nucleus itself. The extracellular matrix is composed of collagen, proteoglycans, water and noncollagenous proteins. The nucleus pulposus is surrounded by a fibrous outer portion, known as the annulus fibrosis, which is composed of cells, collagen fibers and non-fibrillar extracellular matrix. The components of the annulus are arranged in 15-25 lamellae around the nucleus pulposus.

Many men and women around the world suffer from problems in the spinal column. Shooting pains down the leg, called sciatica, that persist for six weeks or so probably mean that nucleus pulposus has leaked through the annulus fibrosus, forming a hernia which is pressing on a nerve.

A herniated disc may bulge out and compress itself onto a nerve, resulting in lower leg pain, loss of muscle control or paralysis. To treat a herniated disc, the offending portions of the disc (i.e., the bulging portions of the nucleus pulposus) are generally removed surgically.

The disc may also be damaged due to disease which typically causes the disc to gradually reduce in height, causing the annulus to buckle, tear or separate, radially and/or circumferentially, and causing persistent and disabling back pain. Degenerative disc disease is generally treated by surgically removing the nucleus pulposus and fusing together the adjacent vertebral bodies so as to stabilize the joint. In the intervertebral disc, however, closure of a tear in the annulus fibrosus does not necessarily prevent further bulging of that disc segment toward the posterior neural elements. Further, there is often no apparent tear in the annulus fibrosus when herniation occurs. Herniation can be a result of a general weakening in the structure of the annulus fibrosus (soft disc) that allows it to bulge posteriorly without a rupture. Furthermore, these procedures ultimately place greater stress on adjacent discs due to their need to compensate for the lack of motion. This may in turn cause premature degeneration of those adjacent discs.

It is therefore recognized that the restoration or replacement of a damaged nucleus pulposus so as to restore the spinal disc to its original configuration and function, may be beneficial to the patient reducing post surgery pain and trauma. With the advent of modern surgery, many new techniques and devices have been developed to restore anatomical structures rather than remove them. Yet, the nucleus pulposus is a sophisticated structure which is difficult to reproduce artificially. It must carry a wide range of different loads, depending on the individual's current activity. By way of example, the nucleus must carry a relatively large load while the individual is carrying a heavy object, yet must accommodate a relatively modest load while the individual is lying down. Furthermore, the nucleus must be able to respond quickly to rapidly changing loads (e.g., while the individual is jumping up and down). The natural nucleus accommodates such load changes by means of an appropriate controlled deformation.

Known in the art are techniques in which a spring is used for applying tension so as to induce growth of soft tissue. Aside from the difficulty of placing a spring within the limited space of the intervertebral disc, the spring induces a continuous displacement of the attached tissues that may be deleterious to the structure and function of the disc. The spring may further allow a posterior bulge in the disc to progress should forces within the disc exceed the tension force applied by the spring.

Recently, techniques for augmenting the intervertebral disc have been proposed. To this end see, e.g., U.S. Pat. Nos. 6,602,291, 6,132,465, 6,110,210, 6,022,376, 5,824,093, 674,295 and 5,562,736, and U.S. Patent Application Nos. 20030040800, 20030195630, 20050085916, 20050203206, 20050222684.

Generally, augmentation is achieved either by fixing implants to surrounding tissues or by relying on the annulus fibrosus to keep them in place. Fixing of implants to surrounding tissues typically includes the replacement of the entire disc. However, by replacing the entire disc, the implant must endure all of the loads that are transferred through that disc space, which, in many cases, exceed those in normal discs. The design of such implant must therefore be extremely robust and yet flexible, qualities which are difficult to obtain simultaneously. An additional drawback of such solutions is that devices that replace the entire disc must be implanted using relatively invasive procedures, normally from an anterior approach. They may also require the removal of considerable amounts of healthy disc material including the anterior annulus fibrosus. Furthermore, these types of implants must be available in many shapes and sizes so as to account for the contour of the neighboring Vertebral bodies which are known to differ from one patient to the other.

Augmentation devices which are not directly fixed to surrounding tissues, are advantageous over the fixed implants, because they do not require the replacement of the entire disc. These devices are typically inserted through a hole in the annulus fibrosus and are inflated, deploy expanding elements or expand, so as to be larger than the hole through which they are inserted. Nucleus implant is often a molded polymer device designed to absorb the compressive forces placed on the spine. For increased strength, the nucleus implant may be combined with an internal matrix of, for example, bio-compatible fibers. The retained annulus fibrosis provides tensile strength. Some desirable attributes of a hypothetical implantable nucleus replacement device include axially compressibility for shock absorbance, excellent durability to avoid future replacement, and bio-compatibility.

Nevertheless, there remains a widely recognized need for, and it would be highly advantageous to have spinal nucleus prosthesis and method for using same, devoid of limitations associated with prior art technologies.

### SUMMARY OF THE INVENTION

According to one aspect of the present invention there is provided an implantable prosthesis device. The prosthesis device comprises one ore more compartnients bounded by a substantially closed porous envelope made at least in part of non-woven polymer fibers, and a filler structure at least partially filling the compartment(s) and being made, at least in part, of non-woven swellable polymer fibers.

According to another aspect of the present invention there is provided a kit for spinal nucleus prosthesis implantation in an annulus fibrosus of an intervertebral disc. The kit comprises the implantable prosthesis device and an insertion device.

According to yet another aspect of the present invention there is provided a method of treating an intervertebral disc having at least an annulus fibrosus. The method comprises advancing the insertion device engaged with an implantable prosthesis device to the annulus fibrosus, disengaging the prosthesis device from the insertion device such that the prosthesis device remains within the annulus fibrosus, and removing the insertion device from the annulus fibrosus.

According to further features in preferred embodiments of the invention described below, the method further comprises mounting the implantable prosthesis on the insertion device.

According to still further features in the described preferred embodiments the method further comprises rolling or folding the implantable prosthesis to a tubular shape prior to the mounting.

According to still further features in the described preferred embodiments the method further comprises imaging the intervertebral disc during the advancing, the disengaging and the removing.

According to still further features in the described preferred embodiments the advancing comprises injecting or extruding the implantable prosthesis device out of the insertion device into the core of the annulus fibrosus.

According to still further features in the described preferred embodiments the method further comprises forming a slit incision in the annulus fibrosus to form an opening in the annulus fibrosus, wherein the advancing the insertion device comprises introducing the insertion device through the opening.

According to still further features in the described preferred embodiments the advancing the insertion device comprises introducing the insertion device through an opening in the annulus fibrosus in a manner such that the prosthesis device is deployed at a contralateral side of the annulus fibrosus cavity, relative to the opening.

According to still further features in the described preferred embodiments the advancing step, disengaging step and removing step are performed such that natural nucleus pulposus of the intervertebral disc remains within the annulus fibrosus.

According to still further features in the described preferred embodiments the insertion device comprises a cannula, for receiving the implantable prosthesis, and an advancing mechanism for delivering the implantable prosthesis from the cannula to the annulus fibrosus.

According to still further features in the described preferred embodiments the swellable polymer fibers comprise water-swellable polymer fibers.

According to still further features in the described preferred embodiments the envelope is made at least in part of polymer fibers.

According to still further features in the described preferred embodiments the envelope is made at least in part of non-woven polymer fibers.

According to still further features in the described preferred embodiments the envelope is made at least in part of woven polymer fibers.

According to still further features in the described preferred embodiments the envelope has an elongated shape characterized by an aspect ratio of at least 5, more preferably at least 10.

According to still further features in the described preferred embodiments the elongated shape is characterized by a diameter which is less than about 2 mm.

According to still further features in the described preferred embodiments the device comprises a plurality of compartments.

According to still further features in the described preferred embodiments the compartments are arranged as a chain of compartments connected by connecting structures.

According to still further features in the described preferred embodiments the compartments are arranged on a polymer filament.

According to still further features in the described preferred embodiments the insertion device is designed and constructed to inject the implantable prosthesis into the annulus fibrosus such that the elongated shape is self-entangled within the annulus fibrosus.

According to still further features in the described preferred embodiments the polymer fibers of the envelope are electrospun polymer fibers.

According to still further features in the described preferred embodiments the polymer fibers of the filler structure are electrospun polymer fibers.

According to still further features in the described preferred embodiments the envelope is characterized by elasticity of at least 10%.

According to still further features in the described preferred embodiments the envelope is characterized by porosity of at least 30%.

According to still further features in the described preferred embodiments the envelope is characterized by water permeability of at least 0.01 ml/cm²/min.

According to still further features in the described preferred embodiments the filler structure comprises at least a first type of layers and a second type of layers, the first type of layers being formed of the swellable polymer fibers, and the second type of layers being formed of polymer fibers incorporated with at least one pharmaceutical agent.

According to still further features in the described preferred embodiments the at least one pharmaceutical agent comprises a growth factor.

According to still further features in the described preferred embodiments the at least one pharmaceutical agent comprises an anti-inflammatory drug.

According to still further features in the described preferred embodiments the at least one pharmaceutical agent comprises an analgesic drug.

According to still further features in the described preferred embodiments the filler structure and/or envelope comprises at least one layer of polymer fibers incorporated with at least one imaging agent.

According to still further features in the described preferred embodiments the device further comprises a support structure disposed so as to provide the envelope with a predetermined shape.

According to still further features in the described preferred embodiments the first type of layers occupies at least 70% of the volume of the compartment(s).

According to still further features in the described preferred embodiments the swellable polymer fibers are selected such that the device swells by at least 250% in volume upon contacting an aqueous medium.

According to still further features in the described preferred embodiments the polymer fibers of the envelope are made of a biostable polymer.

According to still further features in the described preferred embodiments the polymer fibers of the envelope are made of a biodegradable polymer.

According to still further features in the described preferred embodiments the device is foldable or rollable to a dimension of less than about 2 mm in diameter.

The present invention successfully addresses the shortcomings of the presently known configurations by providing a prosthesis device, a kit and a method suitable for treating an annulus fibrosus of a subject.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIGS. 1a-e are schematic illustrations of an implantable prosthesis device, according to various exemplary embodiments of the present invention;

FIG. 2 is a schematic fragment illustration showing a layer of a filler structure of the implantable prosthesis device, in a preferred embodiment in which the layer is formed of a non-woven web of polymer fibers having therein embedded particles constituting a substance;

FIG. 3 is a schematic fragment illustration showing a layer of a filler structure of the implantable prosthesis device, in a preferred embodiment in which the layer is comprises compact objects distributed between the polymer fibers;

FIG. 4 is a schematic illustration of a kit for spinal nucleus prosthesis implantation, according to various exemplary embodiments of the present invention;

FIG. 5 is an image of the implantable prosthesis device in an embodiment in which the device is shaped as a disc;

FIGS. 6a-d are schematic illustrations of a technique for mounting the implantable prosthesis device onto a cannula in the preferred embodiment in which the device has an oval shape; and

FIGS. 7a-b are an image (FIG. 7a) and a schematic illustration (FIG. 7b) of a medical procedure in which the implantable prosthesis device was implanted in a sheep.

### DESCRIPTION OF THE PREFERRED EMBODIMENTS

The present invention is of a device, kit and method which can be used for treating an intervertebral disc of a subject. Specifically, the present invention can be used for replacing or augmenting the nucleus pulposus tissue of the intervertebral disc.

The principles and operation of a device, kit and method according to the present invention may be better understood with reference to the drawings and accompanying descriptions.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

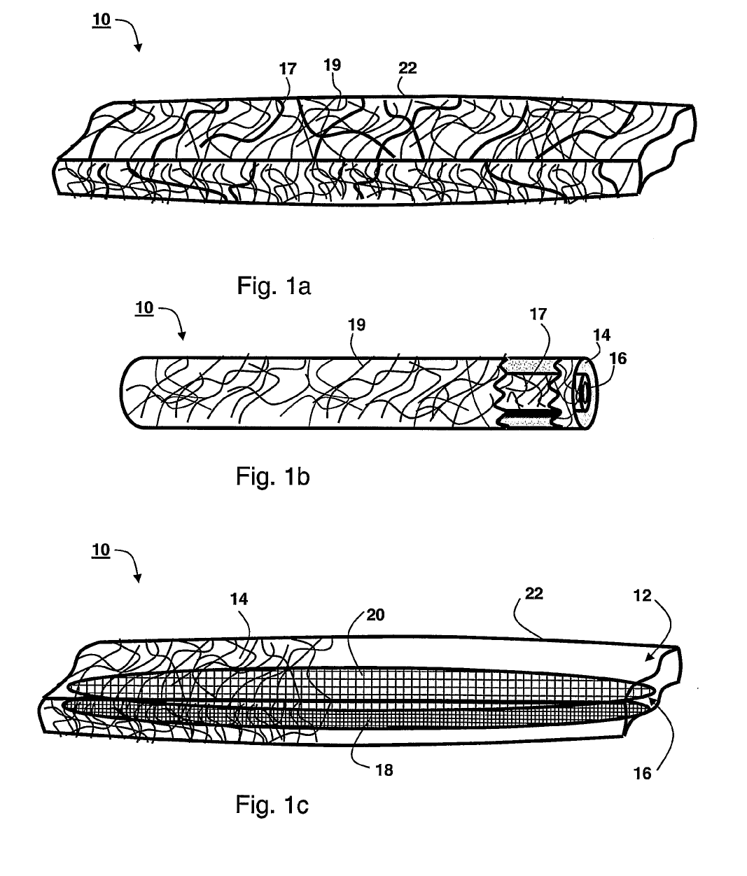

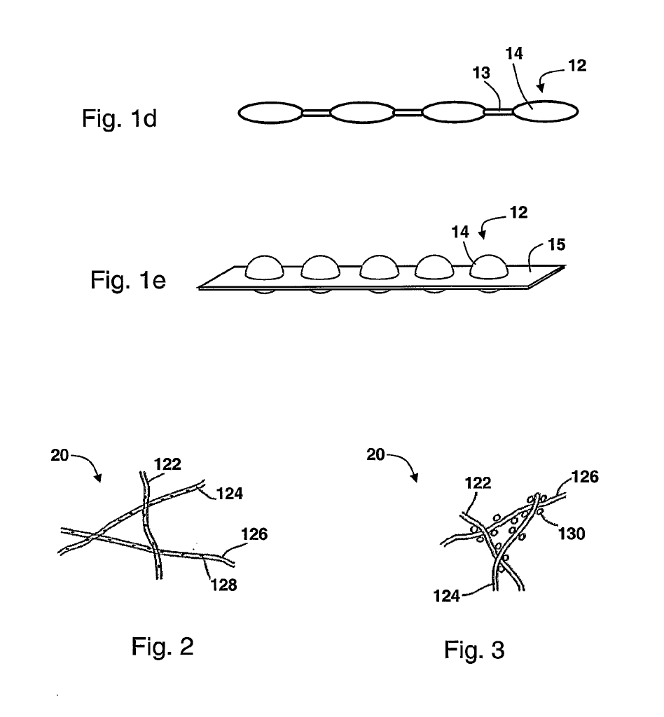

Referring now to the drawings, FIGS. 1a-e illustrates an implantable prosthesis device 10, according to various exemplary embodiments of the present invention.

Device 10 comprises a plurality of non-woven bio-compatible polymer fibers.

The term “polymer”, as used herein includes, but is not limited to, homopolymer, copolymer, e.g., block, graft, random and alternating copolymer, terpolymer, etc., and blends and/or modifications thereof. Furthermore, unless otherwise specifically limited, the term “polymer” includes all possible geometrical configurations, including, without limitation, isotactic, syndiotactic and atactic symmetries.

According to a preferred embodiment of the present invention, two or more types of polymer fibers are incorporated in device 10. The two types of polymer fibers are illustrated in FIG. 1a by different line widths, but it is not to be understood that polymer fibers drawn with thicker or thinner line widths are limited to have larger or smaller cross-section.

A first type of polymer fibers, generally shown at 19, comprises fibers which are preferably elastic so as to allow device 10 to expand in volume. The preferred elasticity of fibers 19 is 10% or more.

Fibers 19 can be made of a biostable or a biodegradable polymer as desired. Biostable polymer suitable for the present embodiments can comprise one or more of the following polymers: thermoplastic polyurethanes, polydimethylsiloxane and other silicone rubbers, polyester, polyolefins, polymethyl-methacrylate, vinyl halide polymer and copolymers, polyvinyl aromatics, polyvinyl esters, polyamides, polyimides and polyethers.

Biodegradable polymer suitable for the present embodiments can comprise one or more of the following polymers: poly(L-lactic acid), poly(lactide-co-glycolide), polycaprolactone, polyphosphate ester, poly(hydroxy-butyrate), poly(glycolic acid), poly(DL-lactic acid), poly(amino acid), cyanocrylate, some copolymers and biomolecules such as collagen, DNA, silk, chitozan and cellulose.

A second type of polymer fibers, generally shown at 17, preferably comprises fibers which are made, at least in part, from a swellable, more preferably water-swellable polymer.

The term “swellable” as used herein describes a functionality of a material to expand or increase in physical size (length, area and/or volume) in the presence of a swelling agent. The term “water-swellable polymer” refers to a swellable polymer for which the swelling agent is aqueous medium, such as, but not limited to, water or biological fluids. A “water-swellable polymer” preferably imbibes water with a concomitant increase in volume when exposed to the aqueous medium. Water swellable polymer generally retains its original identity or physical structure, but in a highly expanded state, during the absorption or adsorption of the water.

In various exemplary embodiments of the invention the swellable polymer is capable, under the most favorable conditions, of absorbing or adsorbing at least about 3 times its weight and, more desirably, at least about 6 times its weight in the presence of the swelling agent.

As used herein the term “about” refers to ±10%.

The water-swellable polymer of the present embodiments can be natural, synthetic or modified natural polymer. In addition, the water-swellable polymer of the present embodiments can be an inorganic polymer, or an organic polymer.

Representative examples of a water-swellable polymers suitable for the present embodiments, include, without limitation, modified polyurethanes such as those marketed under the trade names, Hydrothane™, Hydromed™ and Hydroslip™, polyacrylamide, polyvinyl alcohol, poly(hydroxyethyl methacrylate), poly(hydroxypropyl methacrylate), polyacrylate-polyalcohol and the like.

Water-swellable polymer fibers of the present embodiments can also comprise other materials, such as, but not limited to, poly (isobutylene-co -maleic acide) sodium salt, gelatin and collagen. Since the elasticity of the swellable polymer is typically low, fibers 17 may detached from device 10 upon swelling. Thus, according to a preferred embodiment of the present invention fibers 17 and 19 are preferably interlaced thereamongst, such that friction forces between fibers 19 and themselves and between fibers 19 and fiber 17 secure the structure of device 10. When fibers 17 are swelled, the friction forces substantially prevent fibers 17 from detaching.

FIG. 1b is a fragmentary view illustrating device 10 in a preferred embodiment in which the device comprises more than one layer of polymer fibers. In this embodiment, one or more of the outer layers of device 10 serve as a substantially closed porous envelope 14 for encapsulating a filler structure 16 which according to the presently preferred embodiment of the invention comprises the swellable polymer in the form of non-woven polymer fibers. Thus, fibers 19 are preferably incorporated in one or outer layers and fibers 17 are preferably incorporated in one or more inner layers of device 10. In various exemplary embodiments of the invention envelope 14 comprises polysaccharide, gelatin and/or hydroxypropyl methyl cellulose (HPMC), for enhancing the rigidity of envelope 14.

Although the preferred form of envelope 14 is a non-woven article, this need not necessarily be the case, since, for some applications, it may not be necessary for envelope 14 to be non-woven. For example, envelope 14 can be made of woven fibers or it can be non-fibrous. In any event, envelope 14 is substantially porous and substantially close so as to prevent filler structure for migrating out of device 10 while allowing the swelling agent to penetrate through envelope 14.

In addition to the swellable polymer fibers, filler structure 16 can also comprise biostable and/or biodegradable polymer fibers for increasing the hydrophility of structure 16. Any of the aforementioned types of biostable and/or biodegradable polymer can be used. For example, filler structure 16 can also comprise polymer fibers which are similar to fibers 19 of envelope 14. Envelope 14 and/or filler structure 16 can comprise one or more layers. FIG. 1c is a schematic illustration of device 10 in a preferred embodiment in which the device comprises one or more compartments. Shown in FIG. 1c, is a compartment 12 bounded by envelope 14 (shown partially in FIG. 1c). Compartment 12 is at least partially filled by a filler structure 16, which preferably comprises one or more swellable polymers, and more preferably one or more water-swellable polymers, as further detailed hereinabove.

Filler structure 16 can also comprise a biostable and/or a biodegradable polymer for increasing the hydrophility of structure 14. Representative examples of suitable biostable polymers include, without limitation, thermoplastic polyurethane, polydimethylsiloxane or another type of silicone rubber, polyester, polyolefin, polymethyl-methacrylate, vinyl halide polymer and copolymer, polyvinyl aromatic, polyvinyl ester, polyamide, polyimide and polyether. Representative examples of suitable biodegradable polymers, include, without limitation, poly(L-lactic acid), poly(lactide-co-glycolide), polycaprolactone, polyphosphate ester, poly(hydroxy-butyrate), poly(glycolic acid), poly(DL-lactic acid), poly(amino acid), cyanocrylate, and biolmolecules such as collagen, DNA, silk, chitozan and cellulose derivatives.

The non-woven polymer fibers of envelope 14 and/or filler structure 16 can be formed by any fiber-forming process known in the art. In various exemplary embodiments of the invention a spinning technique is employed. The preferred spinning technique is the electrospinning technique, in which a fine stream or jet of liquid is produced by pulling a small amount of charged liquefied polymer through space using electrical forces. The produced fibers are hardened and collected on a suitably located precipitation device to form the nonwoven article of electrospun fibers. Suitable electrospinning techniques are disclosed, e.g., in International Patent Application, Publication Nos. WO 2002/049535, WO 2002/049536, WO 2002/049536, WO 2002/049678, WO 2002/074189, WO 2002/074190, WO 2002/074191, WO 2005/032400 and WO 2005/065578, the contents of which are hereby incorporated by reference.

It is to be understood that although the according to the presently preferred embodiment of the invention is described with a particular emphasis to the electrospinning technique, it is not intended to limit the scope of the invention to the electrospinning technique. Representative examples of other spinning techniques ;suitable for the present embodiments include, without limitation, a wet spinning technique, a dry spinning technique, a gel spinning technique, a dispersion spinning technique, a reaction spinning technique or a tack spinning technique. Such and other spinning techniques are known in the art and disclosed, e.g., in U.S. Pat. Nos. 3,737,508, 3,950,478, 3,996,321, 4,189,336, 4,402,900, 4,421,707, 4,431,602, 4,557,732, 4,643,657, 4,804,511, 5,002,474, 5,122,329, 5,387,387, 5,667,743, 6,248,273 and 6,252,031 the contents of which are hereby incorporated by reference.

The typical thickness of the polymer fibers of envelope 14 and/or filler structure 16 is, without limitation, from about 50 nm to about 1000 nm, more preferable from 100 nm to 500 nm. The preferred porosity of filler structure 16 is at least 50%, more preferably from about 50% to about 95%, more preferably from about 70% to about 85%. The porosity of envelope 14 is preferably from about 50% to about 70%. Envelope 14 is preferably characterized by water permeability of at least 0.01 ml/cm²/min.

Device 10 can have several basic states. Typically, device 10 has two basic states, referred to herein as the dehydrated state and the swelled state. The term “dehydrated” encompasses both partially and fully dehydration.

In the dehydrated state, device 10 is preferably shaped in such a way that it facilitates insertion into the cavity of the spinal disc through a small incision in the annulus fibrosus. In the dehydrated state, envelope 14 preferably has an elongated shape, which is characterized by an aspect ratio of at least 5, more preferably at least 10, even more preferably at least 20. The elongated shape is preferably, but not obligatorily, convex (e.g., “linguini-style” or “spaghetti-style”). The advantage of the elongated shape is that it can be injected into the cavity of the annulus fibrosus through a small incision or opening such that the envelope is self-entangled within the cavity and remains entrapped therein.

As used herein, the term “aspect ratio” of a structural element such as envelope 14 refers to the length to width ratio of at least one load-bearing surface of the structural element. For example, the aspect ratio of envelope 14 can be calculated by measuring the length and width of the external surface of envelope 14, and dividing the measured length by the measured width. The length and width of a structural element having a rounded circular cross section can be determined by measuring the minor and major diameters of the rounded cross section, respectively. For example, the length and width of the surface can be determined by measuring the length of vectors perpendicular to the perimeter of the surface and extending between two points on the perimeter. The vector having the longest length can be used as the major diameter and the vector having the shortest length can be used as the minor diameter of the surface when calculating the aspect ratio.

The typical dimensions of device 10 when in its dehydrated state are from about 6 mm to about 30 mm in length, from about 0.5 mm to about 4 mm in width and from about 0.5 mm to about 4 mm in thickness. In various exemplary embodiments of the invention the diameter of the device when in its dehydrated state is less than 2 mm. In various exemplary embodiments of the invention the length of the device when in its dehydrated state is about 20 mm.

When device 10 comprises two or more compartments, see, e.g., FIGS. 1d-e, the compartments 12 may be connected chainwise via connecting structures 13 (FIG. 1d) or may be arranged on a polymer filament 15 (FIG. 1e). The envelopes 14 of the compartments can be in a form of spheres, ellipsoids, and the like. The advantage of this embodiment is that such arrangement of the compartments increases the packing factor of device 10 hence better facilitates the deployment of device 10 within the annulus cavity.

Alternatively, envelope 14 can be shaped as a disc. An image of device 10 in the embodiment in which envelope 14 is shaped as a disc is provided in FIG. 5, showing device 10 in its dehydrated state (left) and swelled state (right).

In various exemplary embodiments of the invention device 10 can further comprise a support structure 22 disposed so as to provide envelope 14 with its shape. Support structure 22 is preferably made of a radio opaque wire frame. The frame is preferably made of elastic or a shape memory alloy, such as, but not limited to, Nitinol™. The shape of structure 22 can be generally circular, oval, spherical or it can have any of the shapes described hereinabove.

When device 10 is in the dehydrated state, the shape of envelope 14 can be deformed to a shape in which its cross-section minimized so as to facilitate its insertion through the incision or opening. The deformed shape of envelope 14 is may be that of a folded or rolled shape, such as, but not limited to, the approximate shape of a cylindrical body which length is approximately the length of the longer axis of the nucleus pulposus cross-section. In this embodiment, the device is preferably, but not obligatorily, foldable or rollable to a dimension of less than about 2 mm in diameter. Alternatively, envelope 14 can be injected or extruded through the incision or opening while being in its elongated shape. The advantage of injection or extrusion is that it facilitates the aforementioned self-entanglement and entrapping of device 10 within the cavity of the annulus fibrosus.

In the swelled state, device 10 substantially has the shape of the cavity of the spinal disc created by the partial or complete absence of nucleus pulposus tissue. Device 10 is implanted in the dehydrated state as described above. Once inserted, device 10 imbibes additional water from body fluids and increases its volume until it occupies the cavity. According to a preferred embodiment of the present invention the swellable polymer fibers are selected such that in its swelled state device 10 swells by at least 250% in volume. The typical height of device 10 when in its swelled state is from about 4 mm to about 15 mm.

Device 10 can also have an ex-vivo swelled state, corresponding to the most relaxed polymer network in the state of full hydration of the swellable polymer.

Device 10 changes its state from the dehydrated state to the ex-vivo swelled state in the presence of a swelling agent, such as aqueous medium. Generally, the ex-vivo swelled state is known as the state with minimum enthalpy. In this state, device 10 preferably has a cross-section area and a height which are substantially equivalent or larger than the cross-section area and height of the spinal disc cavity, respectively.

The typical dimensions of device 10 when in its ex-vivo swelled state, are from about 18 mm to about 120 mm in length, from about 3 mm to about 25 mm in width and from about 4 mm to about 15 mm in thickness.

In various exemplary embodiments of the invention filler structure 16 comprises one or more layers of one or more types. In the representative example illustrated in FIG. 1 there are two layers, designated by reference numerals 18 and 20. Layer 18 is preferably formed of swellable polymer fibers, and layer 20 is preferably formed of polymer fibers incorporated with at least one substance, such as, but not limited to, a pharmaceutical agent, a medicament, an imaging agent and a biological material. Filler structure 16 can include more than two layers, for example, a plurality of layers formed of swellable polymer fibers and/or a plurality of layers formed of polymer fibers incorporated with one or more substances. When more than one layer is incorporated with a substance, different such layers can be incorporated with different substances. In various exemplary embodiments of the invention layer(s) 18 occupy at least 70% of the volume of compartment(s) 12, when device 10 is in the dehydrated state.

Representative examples of substances which can be incorporated in layers 20, include, without limitation, growth factors (particularly, but not exclusively, recombinant human protein), anti-inflammatory drugs, antibiotics, analgesics (particularly, but not exclusively, long acting analgesic compounds), immunosuppressive agents and any combination or sub-combination thereof. For example, layers 20 can be incorporated with transforming growth factor beta, bone morphogenetic proteins, fibroblast growth factors, platelet-derived growth factors, insulin-like growth factors or the like. Also contemplated are radiopaque agents, MRI or ultrasound contrast agents, radio-labeled agents and the like.

The substance(s) may be incorporated into the fibers of layer 20 in more than one way. For example, when the fibers are formed via a spinning technique, the substance(s) can be mixed with the liquefied polymer used in the spinning process.

FIG. 2 illustrates another technique for incorporating the substances into layer 20. Shown in FIG. 2 is a portion of a non-woven web of polymer fibers produced according to a preferred embodiment of the present invention. Fibers 122, 124 and 126 intersect and are joined together at the intersections, the resultant interstices rendering the web highly porous. The fibers are preferably, thin so as provide layer 20 with a large surface area which allows a high quantity of substances to be incorporated thereon. When the fibers are electrospun fibers, their surface area approaches that of activated carbon, thereby making the non-woven web of polymer fibers an efficient local drug delivery system. In the representative illustration shown in FIG. 2, the substance(s) are constituted by particles 128 embedded in the polymer fibers. This embodiment is particularly useful when the substance is a medicament which is to be released during the first post-operative days and weeks. The duration of the delivery process is effected by the type of polymer used for fabricating the layer. Specifically, optimal release rate is ensured by using moderately stable biodegradable polymers.

FIG. 3 illustrates an alternative technique for incorporating the substance(s) into layer 20. In this embodiment, the substance(s) are constituted by compact objects 130, distributed between the polymer fibers of the layer. Compact objects 130 may be in any known form, such as, but not limited to, moderately stable biodegradable polymer capsules.

When it is desired to incorporate a substance which is released over a prolong period of time (e.g., several months to several years) the substance(s) are dissolved or encapsulated in a layer made of biostable fibers. The rate diffusion from within a biostable layer is substantially slower, thereby ensuring a prolonged effect of release.

Thus, the time scale of substance release is controlled according to various exemplary embodiments of the present invention by the type of polymer, the technique in which the substance(s) are introduced into the polymer fibers, and the concentration of the substance.

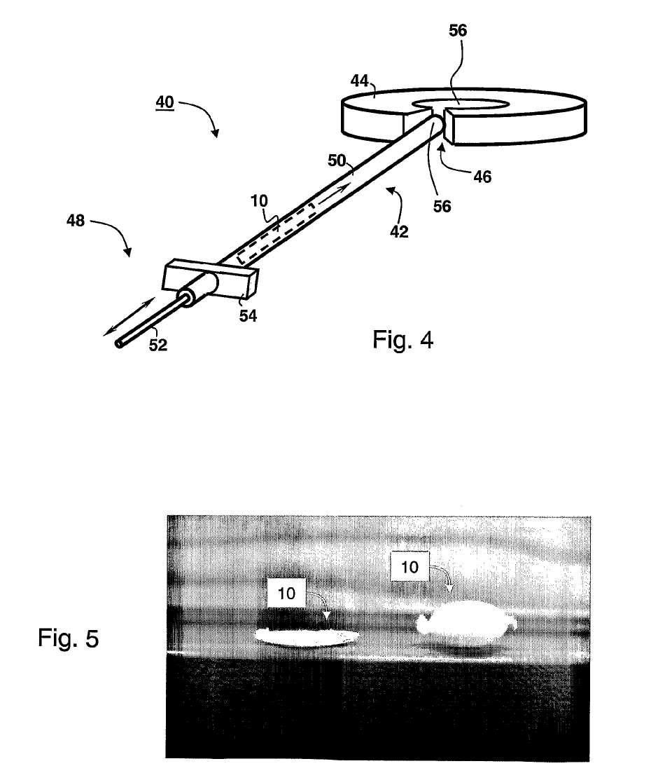

Reference is now made to FIG. 4, which is a schematic illustration of a kit 40 for spinal nucleus prosthesis implantation. Kit 40 comprises implantable prosthesis device 10 as described above, and an insertion device 42, for advancing device 10 into the annulus fibrosus 44 of the subject. Insertion device 42 is typically manufactured as a cannula 50 or the like, having an outer diameter of about 2-3 mm, which is sizewise compatible with the dimensions of a typical opening or incision 46 in annulus fibrosus 44. The inner diameter of cannula 50 is typically about 0.1-0.2 mm smaller than the outer diameter. Insertion device 42 preferably comprises a mechanism 48 for advancing device 10 within cannula 50, which may comprise a rod and/or a movable handle 52. Mechanism 48 may also comprise an extruder 54 for extruding implantable device 10 into cannula 50. The tip 56 of cannula 50 may tapered to ease the insertion of cannula into opening or incision 46. Cannula 50 may be formed of any of the known cannula materials such as plastic or metal. Device 42 may be a single use device or may be reusable. Device 42 of the present embodiments is intended for insertion of implantable device 10 in animals including humans, livestock and the like.

Insertion device 42 can be provided with implantable prosthesis device 10 engaged thereon, or it can be provided in a separate sterile package, in which case device 10 is mounted onto insertion device 42 in the operating room prior to the procedure. The mounting can be done either directly into cannula 50 (e.g., by means of rod 52) or by introducing device 10 into extruder 54.

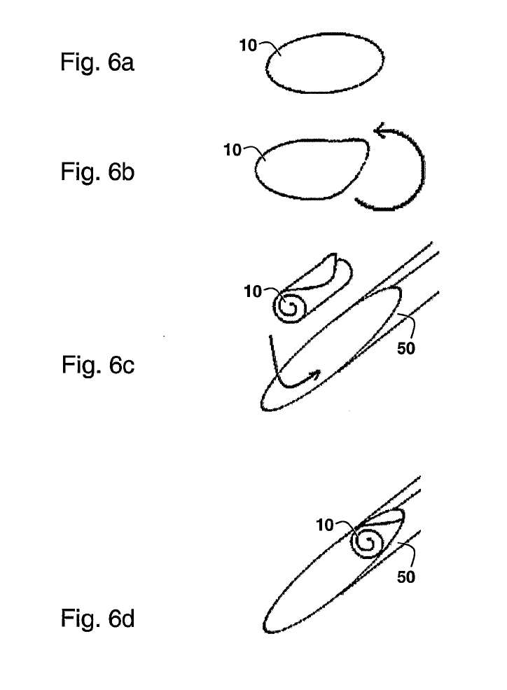

An exemplified technique for mounting device 10 onto cannula 50 in the preferred embodiment in which device 10 has an oval shape is illustrated in FIGS. 6a-d. In this technique, device 10 is first rolled or folded to reduce its dimensions. Subsequently device 10 is introduced into cannula 50 while being in its rolled or folded state. Shown in FIGS. 6a-d is device 10 in its relaxed state outside the body (FIG. 6a), at the beginning of its rolled state (FIG. 6b), in its final rolled state before (FIG. 6c) and after (FIG. 6d) the introduction of device 10 into cannula 50.

In use, the surgeon advances insertion device 42 and implantable prosthesis device 10 to annulus fibrosus 44, disengages device 10 from insertion device 42 such that device 10 remains within the core 56 of annulus fibrosus 44, and removes insertion device 42 from annulus fibrosus 44. When device 10 has an elongated shape, it is preferably injected or extruded out of cannula 50 to facilitate its self entanglement within core 56. Once insertion device 42 is removed, the self entanglement prevents device 10 from exiting core 56 through opening or incision 46.

Device 10 is typically implanted into an annulus fibrosus which is at least partially damaged. Specifically, device 10 can be used for augmentation and/or replacement of the nucleus pulposus in the annulus fibrosus of the subject. Thus, when device 10 is used for augmentation, device 10 is preferably implanted in a partially damaged annulus fibrosus in which more than 50% of the natural nucleus pulposus is still present in its core. When device 10 is used for nucleus pulposus replacement, the implantation is performed in an annulus fibrosus in which less than 50% of the natural nucleus pulposus is present in its core.

The implantation can also comprise an incising procedure in which the surgeon forms a slit incision (see 46 in FIG. 4) in the annulus fibrosus to allow the cannula to access the vacant cavity in its core. Alternatively, if the annulus fibrosus is damaged such that it already has an opening, the insertion device can engage the opening without performing a surgical incision. The implantation can also comprise a nucleus pulposus removal procedure in which part or all the nucleus pulposus is surgically removed to create cavity of cross section approximating the cross section of device 10. Device 10 can then be implanted in the vacated cavity.

In various exemplary embodiments of the invention the implantation procedure of the prosthesis device is performed by an image guided procedure. In these embodiments, the envelope and/or filler structure preferably comprises an imaging agent as described hereinabove. Thus, the treated intervertebral disc and optionally one or more nearby discs are imaged using, e.g., MRI, ultrasound, CT, X-ray, gamma rays or the like, during one or more steps of the operation.

Once device 10 is in place, the filler structure swells. The preferred swelling is spontaneous swelling whereby the swellable polymer fibers of the filler structure imbibe water from biological fluids. Alternatively or additionally, a swelling agent can be injected into the core of the annulus fibrosus to facilitate swelling.

Following implantation, the incision can be secured by suturing or using biocompatible glue. Once the filler structure swells for a period of about 10-15 minutes, the prosthesis increases in height until it runs against the vertebral end plates. The continuing swelling increases vertebral separation and stretches the annulus pulposus into the shape and tension required for its long-term function. The swelling capability of the implantable prosthesis device of the present embodiments protects the annulus fibrosus against excessive radial swelling pressure that may lead to herniation or extrusion of the natural nucleus pulposus or the prosthesis device itself. The prosthesis device of the present embodiments becomes party hydrated and substantially conforms the shape of the annulus fibrosus core. The dimensions of the device in its indwelling state vary from one subject to the other. Typically, but not obligatorily, when the device is in its indwelling state its cross section is from about 10 mm² to about 30 mm², and its height is from about 2 mm to about 8 mm.

The kit of the present embodiments may, if desired, be presented in a pack which may contain one or more units of the implantable prosthesis device and/or one or more units of the insertion device. The pack may be accompanied by instructions for implantation. The pack may also be accompanied by a notice in a form prescribed by a governmental agency regulating the manufacture, use, or sale of implantable devices, which notice is reflective of approval by the agency of the form of the compositions for human or veterinary implantation procedures. Such notice, for example, may include labeling approved by the U.S. Food and Drug Administration.

Additional objects, advantages and novel features of the present invention will become apparent to one ordinarily skilled in the art upon examination of the following example, which is not intended to be limiting. Additionally, each of the various embodiments and aspects of the present invention as delineated hereinabove and as claimed in the claims section below finds experimental support in the following example.

### Example

Reference is now made to the following example, which together with the above descriptions illustrates the invention in a non limiting fashion.

### Implantation of Prosthesis Device in Sheep

### Introduction

The purpose of the present study is to evaluate the performance and the local tolerance of the implantable prosthesis device of the present embodiments in sheep. Two sheep (strain Grivette, about 60 Kg in weight and about 4 years of age) were included in the study.

The study was conducted in accordance with the requirements of the FDA Good Laboratory Practice (GLP) Regulations, 21 CFR 58 revised as of Apr. 1, 2005 and the “Bonnes Pratiques de Laboratoire” (BPL), arrêté du 14 mars 2000 described in the “Journal Officiel du 23 Mars 2000.” The experiment was performed at the BIOMATECH S.A.S. test facilities, France.

### Objectives

The objective of the present study was to determine the effects of the implantable prosthesis device of the present embodiments on soft and hard tissue following functional use in an animal model.

The implantable prosthesis device had an elongated rod-like shape, 4 mm in length, and 2.5 mm in diameter. The filler structure was incorporated with a dexamethasone for reducing or preventing inflammation and barium sulfate for providing the device with enhanced radiopacity.

### Procedure

Prior to the surgical procedure, the animals were fasted overnight. At the time of implantation, pre-medication and anesthesia was performed by intravenous injection of thiopental-pentobarbital mixture (NESDONAL®, MERIAL, France; Pentobarbital sodique, CEVA Santé animale, France) and atropine (Atropinum sulfuricum, AGUETTANT, France) followed by inhalation of a O₂-isoflurane (1-4%) mixture. The lumbar area was clipped free of fur. The skin was scrubbed with povidoine iodine (VETEDINE®, VETOQUINOL, France).

Cardiac monitoring was performed during surgery. Each animal was tied in a lateral decubitus position. Using standard aseptic surgical technique, a right retroperitoneal approach to the lumbar spine was conducted. The interval between the psoas major and psoas minor was identified and separated. Two contiguous intervertebral discs (L₄-L₅ and L₅-L₆) were identified and exposed. Care was taken not to interrupt the outer annular fibers of the disc during exposure. A full thickness incision was made through the annulus fibrous and into the nucleus pulposus. The annular incisions were made on the right anterolateral aspect of the disc. A straight 2 mm transverse slit was made parallel to the vertebral end plates. Care was taken to preserve the annular tissue in the slit incision.

The nucleus prosthesis device of the present embodiments was injected through a cannula of outer diameter 2.8 mm and inner diameter 2.7 mm towards the opposite, contralateral edge of the nucleus. The cannula was advanced into the nucleus cavity space until the contralateral edge was sensed, due to the difference in piercing resistances upon contacting the contralateral edge. A solid stainless steel rod was used as an advancing mechanism to advance the prosthesis device within the cannula toward the contralateral edge. The annular fibers of the incision were closed using a simple suturing technique and 3-0 Vicryl suture, while keeping the prosthesis device within the nucleolus cavity. No ruptured concurred in the prosthesis devices throughout the procedure. Subcutaneous and cutaneous tissues were closed using standard procedures. The wound was disinfected using an iodine solution and a dressing was applied.

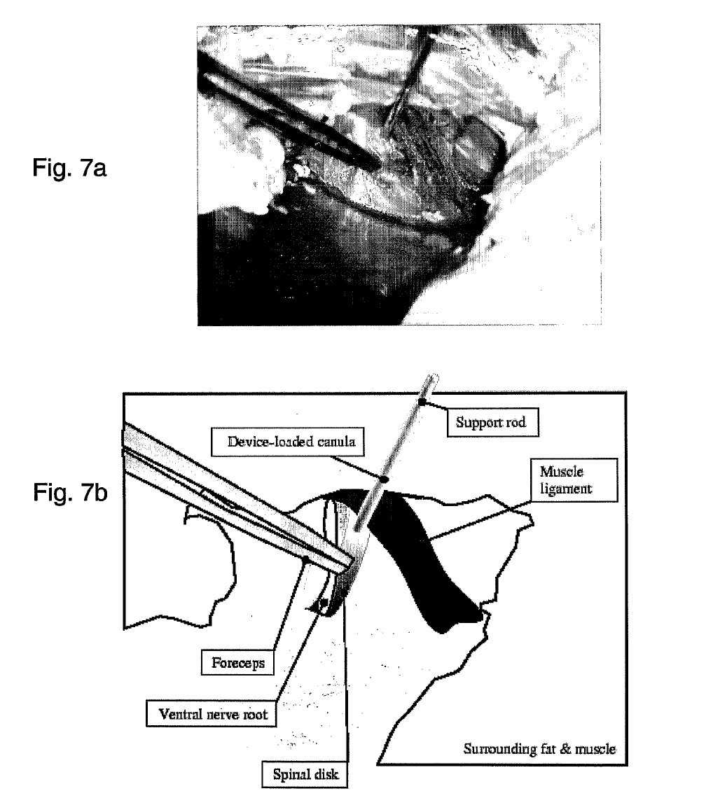

FIGS. 7a-b are an image (FIG. 7a) and schematic illustration (FIG. 7b) of the medical procedure, showing the surgeon forceps holding the spinal disc while protecting the ventral nerve root and the cannula with the stainless steel rod while being advanced into the into the nucleus cavity space behind the muscle ligament.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

1-45. (canceled)

46. An implantable prosthesis device, comprising at least one compartment bounded by a substantially closed porous envelope, and a filler structure at least partially filling said compartment, said envelope and/or said filler structure being made at least in part of electrospun swellable polymer fibers wherein said swellable polymer fibers comprise water-swellable polymer fibers and said fibers are either woven or not.

47. An implantable prosthesis device, comprising non-woven swellable fibers made at least in part of a swellable polymer, and non-woven elastic fibers made of at least one polymer other than said swellable polymer, said swellable fibers being interlaced with said elastic fibers.

48. The device according to claim 47 wherein said envelope has an elongated shape characterized by an aspect ratio of at least 5 and a diameter which is less than about 2 mm further wherein said at least one compartment comprises a plurality of compartments, said plurality of compartments arranged as a chain of compartments connected by connecting structures, further wherein said plurality of compartments are arranged on a polymer filament.

49. A kit for spinal nucleus prosthesis implantation in an annulus fibrosus of an intervertebral disc, comprising: an implantable prosthesis device, and an insertion device, wherein said prosthesis device comprises at least one compartment bounded by a substantially closed porous envelope, and a filler structure at least partially filling said compartment and being made at least in part of non-woven swellable polymer; further wherein said non-woven swellable polymer optionally comprises non- woven elastic fibers made of at least one polymer other than said swellable polymer, said swellable fibers being interlaced with said elastic fibers further wherein said insertion device comprises a cannula, for receiving said implantable prosthesis, further wherein said insertion device is adapted to inject said implantable prosthesis into the annulus fibrosus such that said elongated shape is self-entangled within the annulus fibrosus and an advancing mechanism for delivering said implantable prosthesis from the cannula to the annulus fibrosus.

50. A method of treating an intervertebral disc having at least an annulus fibrosus, said method comprising steps of:
obtaining said prosthesis device having at least one compartment bounded by a substantially closed porous envelope, and a filler structure at least partially filling said compartment and being made at least in part of non-woven swellable polymer fibers;
rolling or folding said implantable prosthesis to a tubular shape;
obtaining said insertion device;
engaging said insertion device with said prosthesis device;
mounting said implantable prosthesis on said insertion device;
advancing said insertion device engaged with said implantable prosthesis device to the annulus fibrosus, optionally injecting or extruding said implantable prosthesis device out of the insertion device into a core of the annulus fibrosus; and
imaging the intervertebral disc during said advancing, said disengaging and said removing.

51. The method of claim 49, further comprising forming a slit incision in the annulus fibrosus to form an opening in the annulus fibrosus, wherein said advancing said insertion device comprises introducing said insertion device through said opening and said advancing further comprises introducing said insertion device through an opening in the annulus fibrosus in a manner such that said prosthesis device is deployed at a contralateral side of the annulus fibrosus cavity, relative to said opening.

52. The method of claim 50 wherein said advancing, said disengaging and said removing is performed such that natural nucleus pulposus of the intervertebral disc remains within the annulus fibrosus.

53. The device, according to claim 46 characterised in that said non-woven elastic fibers have an elasticity of at least 10%, and further wherein said envelope is defined by at least one property selected from the group consisting of an elasticity of at least 10%, porosity of at least 30% water permeability of at least 0.01 ml/cm2/min and foldable or rollable to a dimension of less than about 2 mm in diameter.

54. The device, according to claim 46, wherein said filler structure comprises at least a first type of layers and a second type of layers, said first type of layers being formed of said swellable polymer fibers, and said second type of layers being formed of polymer fibers incorporated with at least one pharmaceutical agent.

55. The device, according to claim 54 wherein said at least one pharmaceutical agent comprises at least one selected form the group consisting of growth factor, anti-inflammatory drug and analgesic drug.

56. The device, according to claim 46, wherein at least one of said filler structure and said envelope comprises at least one imaging agent incorporated within, preferably in said elastic fibers and/or said swellable fibers.

57. The device according to claim 46, wherein said device further comprises a support structure disposed so as to provide said envelope with a predetermined shape and further wherein said first type of layers occupies at least 70% of the volume of said at least one compartment.

58. The device according to claim 46 wherein said device further comprises swellable polymer fibers adapted to facilitate swelling of said device by at least 250% in volume upon contacting an aqueous medium further wherein said polymer fibers of said envelope are made of a biostable polymer or biodegradable polymer.
